SEQUENCE LISTING (3 sequences)

SEQ ID NO: 1 - protein, 12 aa, Artificial
  synthetic construct
ILRWPWWPWRRK

SEQ ID NO: 2 - protein, 7 aa, Artificial
  synthetic construct
WWPWRRK

SEQ ID NO: 3 - protein, 4 aa, Artificial
  synthetic construct
WRRK

(19) United States
(12) Patent Application Publication
(10) Pub. No.: US 2010/0197891 A1
(43) Pub. Date: Aug. 5, 2010

(54) METHOD FOR PEPTIDE SYNTHESIS

(76) Inventors: Matthieu Giraud, Sion (CH); Fernando Albericio, Barcelona (ES); Francesca Quattrini, Zurich (CH); Oleg Werbitzky, Veyras (CH); Katja Senn, Visp (CH); Michaela Williner, Brig-Glis (CH)

Correspondence Address: HOFFMANN & BARON, LLP, 6900 JERICHO TURNPIKE, SYOSSET, NY, 11791, US

(21) Appl. No.: 12/444,408

(22) PCT Filed: Oct. 3, 2007
(86) PCT No.: PCT/EP2007/008581

§ 371 Date: Mar. 16, 2010

(30) Foreign Application Priority Data
Oct. 5, 2006 (EP) EP06020893

(51) Int. Cl. C07K 1/04; C07K 7/08

(52) U.S. Cl. 530/327; 530/334

(57) ABSTRACT

A new method of anchoring a growing peptide chain during chemical synthesis to a solid-phase support is devised. Novel amino acid derivatives and peptide derivatives, both unbonded and bonded to a solid-phase support, are also provided.

The present invention relates to the field of solid-phase peptide synthesis, and in particular to an improved method for building peptide chains by attaching protected amino acids such as Fmoc-amino acids to the free N-terminus of a growing peptide in solid-phase synthesis.

It is known that bulky amino acids and in particular arginine, homo- and norarginine are much more difficult to couple to a growing peptide chain than other amino acids. The problem occurs with most commonly used coupling reagents and is even more severe in solid-phase synthesis, where additional spatial restraints may be caused by the resin surface. Commonly, this problem is sought to overcome by using a higher than standard amount of the amino acid or oligopeptide to be coupled, and in particular by repeated coupling cycles. Of course, this approach of using several reagents in excess results in considerable wasting of valuable reagent.

Di Bello et al. (Semisynth. Pept. Proteins, Pap. Int. Meet. Protein Semisynth., meeting date 1977, “coupling of arginine peptides”, 1978, 373-379) examined this problem using standard N,N′-dicyclohexylcarbodiimide (DCC) coupling chemistry, detailing experimental data and testing different coupling auxiliaries for coupling either the dipeptide Z-Phe-Arg-OH or Boc-Glu(OBzl)-Arg-OH to the N-terminus of a protected penta- or nonamer attached to Merrifield polystyrene resin. All couplings were conducted at room temperature. The best results were obtained using a mixture of the coupling reagent DCC and the additive 1-hydroxy-benzotriazole (HOBt) resulting in quantitative yield. As a disadvantage, this method necessitated repeated coupling cycles at 5:1 molar ratio of dipeptide and oligomer in N,N-dimethylformamide.

However, despite the use of HOBt, comparatively high levels of racemisation remained a serious drawback of carbodiimide coupling reagents leading later to the development of entirely different coupling reagents such as O-(benzotriazol-1-yl)-N,N,N′,N′-tetramethyluronium hexafluorophosphate (HBTU) or the benzotriazol-1-yloxy-tris(dimethylamino)phosphonium hexafluorophosphate (BOP) series of reagents as reviewed in Jiang et al., Tetrahedron 1998, 54, 14233-14254.

According to Nishimura et al., Chem. Pharm. Bull. 1976, 24, 1568-1575, the problem of coupling bulky arginine or homoarginine can be solved by e.g. coupling much less sterically demanding ornithine to the peptide chain and later, i.e. after the last coupling step and before global deprotection, converting said ornithine into arginine by guanidation.

As a disadvantage, this approach involves additional chemical reaction steps which raise the impurity level. Another negative effect is the need of a challenging protection strategy as ornithine must be blocked by an orthogonal protecting group that can be specifically cleaved off as needed for the subsequent guanidation. Finally, guanidation is performed after cleavage from the resin. However, traditional polystyrene based resins such as Wang resin or Rink resin usually require rigorous cleavage conditions leading to undesired partial or complete deprotection of the peptide.

Consequently, there is a high need for a method to couple bulky amino acids with a lysine residue or its homologues so that peptides comprising such a challenging sequence motif are accessible in commercial scale. One peptide of interest is the anti-infective, cationic “indolicidin” Ile-Leu-Arg-Trp-Pro-Trp-Trp-Pro-Trp-Arg-Arg-Lys-NH₂ which shows antimicrobial and antibacterial activity. Such cationic, anti-infective peptides are in general more active due to their C-terminally amidated form. US-A1-2003/0219854 discloses indolicidin and its further derivatives as a new class of broad-spectrum antimicrobial substances which may help to combat the rapid spread of multiple drug resistance towards standard antibiotics amongst pathogenic microbes. The sequence of indolicidin presents a real challenge to achieve acceptable coupling yield as two arginine residues have to be subsequently coupled to a first lysine.

Therefore, it is an object of the present invention to devise a method for overcoming the coupling problem with arginine residues or the like in solid-phase peptide synthesis, especially when coupling arginine or its homologues to a sterically equally demanding lysine or lysine homologue. According to the present invention, the problem of low coupling efficiency is surprisingly solved by applying a side chain anchoring strategy. The present invention results in strongly improved coupling yields that avoid undesired early chain termination in solid-phase synthesis.

The object described above is achieved by the method of claim 1. Compounds derived when applying the method of claim 1 are also objects of this invention.

Applicants have found a method for peptide synthesis starting from a compound of formula

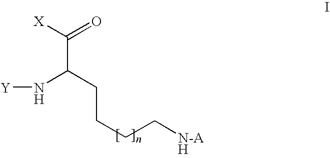

wherein A is a solid-phase support or a linker grafted to a solid-phase support; n is an integer between zero and ten; X is C₁₋₆ alkoxy, aryl-substituted C₁₋₆ alkoxy, aryloxy, allyloxy, an optionally protected amino acid residue, an optionally protected peptide residue or NR¹R², wherein R¹ and R² are independently hydrogen or C₁₋₁₀ alkyl; and Y is a protecting group being orthogonal to the bond between A and the amino function; and comprising the steps of
   (a) deprotecting the N-terminal α-amino function,
   (b) coupling an at least N-terminally protected amino acid or peptide having a free or activated carboxylic acid function with the deprotected α-amino function of step (a), thus elongating the compound of formula I,
   (c) optionally repeating at least once steps (a) and (b), wherein the at least N-terminally protected amino acid or peptide is identical or different to that of the preceding step (b),
   (d) cleaving the resulting peptide from A,
   (e) optionally removing all protecting groups which remained after step (d),
   (f) isolating and optionally purifying the peptide thus obtained.

Here and as follows, the term “C₁₋ₙ alkyl” is to be understood to mean any linear or branched alkyl group containing 1 to n carbon atoms. For example, the term “C₁₋₆ alkyl” comprises groups such as methyl, ethyl, propyl, isopropyl, butyl, isobutyl, sec-butyl, tert-butyl, pentyl, isopentyl (3-methylbutyl), neopentyl (2,2-dimethylpropyl), hexyl, isohexyl (4-methylpentyl) and the like.

Accordingly, the term “C₁₋ₙ alkoxy” means a group composed of a C₁₋ₙ alkyl group as defined above and an oxygen atom linked by a single covalent bond.

The term “aryl-substituted C₁₋ₙ alkyl” is to be understood to mean a group composed of a C₁₋ₙ alkyl group as defined above which is substituted at any position of the linear or branched carbon chain with at least one phenyl group. The phenyl group may be optionally substituted with at least one substituent selected from the group consisting of hydroxyl, C₁₋₂ alkoxy and halogen. Examples of aryl-substituted C₁₋ₙ alkyl groups are benzyl, 1-(3-hydroxyphenyl)-propane-2-yl or 1-(3-methoxyphenyl)propane-2-yl.

Here and as follows, the term “allyloxy” is to be understood to mean an allyl group which may be optionally substituted by C₁₋₃ alkyl or halogen.

In a preferred embodiment of the invented method, Y of the compound of formula I is an orthogonal protecting group selected from the group consisting of Fmoc, Boc, Cbz, Npys and Alloc; with the proviso that Y is not Alloc if X is allyloxy.

Here and as follows, “Fmoc” abbreviates fluoren-9-ylmethoxycarbonyl, “Boc” abbreviates tert-butyloxycarbonyl, “Cbz” abbreviates benzyloxycarbonyl, “Npys” abbreviates 3-nitro-2-pyridenesulfenyl and “Alloc” abbreviates allyloxycarbonyl.

Here and as follows, the term “orthogonal” related to two different protecting groups is to be understood to mean that one protecting group is removable whilst the other remains stable under the same reaction conditions.

Accordingly, the term “orthogonal” related to a protecting group and a bond between the amino function of the lysine side chain or its homologues and the solid-phase support or linker grafted to a solid-phase support A is to be understood to mean that the protecting group is removable whilst said bond remains stable under the same reaction conditions.

The peptide according to the present invention may be any peptide comprising natural or non-natural amino acids and if chiral, in its L or D configuration or as racemate. Examples of non-natural amino acids are homocysteine, homoarginine, cyclohexylalanine, penicillinamide (Pen) or ornithine (Orn).

The terms “peptide backbone”, “main chain”, “side chain” and the prefixes “nor” and “homo” are construed in the present context in accordance to the IUPAC-IUB definitions (Joint IUPAC-IUB Commission on Biochemical Nomenclature, “Nomenclature and Symbolism for Amino Acids and Peptides”, Pure Appl. Chem. 1984, 56, 595-624).

In its wider meaning, “homo” is to be understood to mean up to nine extra methylene groups added in a linear fashion to the lysine side chain. In its narrower and preferred meaning, “homo” amounts to just one extra methylene group in the side chain. “Nor” is always construed in the present context as to amount to one intermittent methylene group been eliminated from the side chain of natural ε-lysine.

In the present context, the “ω-amino group” of an amino acid side chain is to be understood to mean the “terminal” amino group of the side chain irrespective of the carbon chain length.

In a preferred embodiment of the invented method, n of the compound of formula I is zero, one, two, three, four, five, six, seven eight, nine, ten; preferably n is zero, one, two, three, four; i.e. the amino acid residue anchored through its amino side chain is ε-lysyl, ω-homolysyl or ω-norlysyl.

In an also preferred embodiment of the invented method, R¹ and R² of the compound of formula I are independently hydrogen, methyl, ethyl, propyl and butyl; preferably hydrogen, methyl and ethyl; and most preferably hydrogen.

In a preferred embodiment, the N-terminally protected amino acid of step (b) is N-terminally protected arginine (Arg) or homoarginine (Har). In an also preferred embodiment, the N-terminally protected peptide of step (b) contains Arg or Har as C-terminal residue.

The guanidino group of Arg or Har may be protected or unprotected. Any kind of suitable guanidino protecting groups known to the skilled person may be used, such as Cbz, 2,3,6-trimethyl-4-methoxybenzenesulfonyl (Mtr), nitro, tosyl, 5-sulfonyl-2,2,4,6,7-pentamethyl-benzofuran (Pbf), 2,2,5,7,8-pentamethylchroman-6-sulfonyl (Pmc), adamantyloxycarbonyl, tert-butyloxycarbonyl (Boc) or trityl (Trt).

In a more preferred embodiment, the Arg or Har side chain is protected by Pbf.

According to the present invention, it is another preferred embodiment to couple Arg or Har, preferably when used as Fmoc-Arg or Fmoc-Har, without a covalently attached guanidino protecting group but applying non-covalent protection chemistry. This may be achieved by ensuring that after coupling of the individual Arg or Har residue, the guanidino group is quantitatively protonated prior to any further coupling reactions, thus forming a stable ion pair with the proton donor in organic solvent. In practice, the resin-bonded peptide may be treated with an excess of the acidic coupling auxiliary such as 1-hydroxybenzotriazole (HOBt), benzotriazine derivatives or azabenzotriazines which may be further substituted on the aromatic core. Another possibility of scavenging the charge of the guanidinium group is to use tetraphenylborate as counter ion for e.g. protonated Fmoc-protected Har as set forth in U.S. Pat. No. 4,954,616.

It is well known in art that in solid-phase synthesis most of, or preferably all functional groups in amino acid side chains must be masked with permanent protecting groups that are not affected by the reaction conditions employed during peptide chain assembly. The α-amino group of each amino acid to couple is temporarily protected with a protecting group that is preferably orthogonal to the side chain protecting groups, except for the last amino acid to couple, which can be removed using the same deblocking chemistry as the side chain protecting groups. After side chain anchoring of the first amino acid as set forth in the present invention, the temporary α-amino protecting group is removed.

All suitable protecting groups known in the art may be used for both protecting the side chain functions and the α-amino group of the amino acids or peptides used in steps (b) and (c) of the present invention. Suitable protecting groups include but are not limited to fluoren-9-ylmethoxycarbonyl (Fmoc), benzyloxycarbonyl (Cbz), tert-butyloxycarbonyl (Boc), 2-(4-biphenyl)-1)-isopropyloxycarbonyl (Bpoc), acetamidomethyl (Acm), acetyl (Ac), allyl (All), allyloxy-carbonyl (Alloc), benzoyl (Bz), benzyl (Bzl), 3-carboxypropanoyl (Suc), 5-sulfonyl-2,2,4,6,7-pentamethylbenzofuran (Pbf) and trityl (Trt).

In a preferred embodiment of the invented method, Y of the compound of formula I is Fmoc and the N-terminally protected amino acids or peptides of steps (b) and (c) are Fmoc-protected, except for the N-terminally protected amino acid or peptide of the lastly repeated step (c), which is protected by an protecting group being orthogonal to Fmoc, preferably being Boc.

In a preferred embodiment of the invented method, Y of the compound of formula I is Alloc and the N-terminally protected amino acids or peptides of steps (b) and (c) are Fmoc-protected, except for the N-terminally protected amino acid or peptide of the lastly repeated step (c), which is protected by an protecting group being orthogonal to Fmoc, preferably being Boc.

Coupling reagents, coupling additives and aprotic, polar solvents such as e.g. dimethylformamide or N-methylpyrrolidone, or mixtures thereof, are well known in the art and are described e.g. in Bodanszky, “Principles of Peptide Synthesis”, 2^{nd} ed., Springer Verlag, 1993). Examples for coupling reagents are diisopropylcarbodiimide (DIC), 1,3-dicyclohexylcarbodiimide (DCC), N-ethyl-N′-(3-dimethylaminopropyl)-carbodiimide (EDC), benzotriazol-1-yloxy-tripyrrolidinophosphonium hexafluorophosphate (PyBOB), O-(1H-benzotriazol-1-yl)-N,N,N′,N′-tetramethyluronium tetrafluoroborate (HBTU) and O-(1H-6-chlorobenzotriazol-1-yl)-N,N,N′,N′-tetramethyluronium tetrafluoroborate (TCTU). Examples for coupling additives are N-hydroxybenzotriazole (HOBt), 6-chloro-N-hydroxybenzotriazole (6-chloro-HOBt), N-hydroxysuccinimide and N-hydroxy-3,4-dihydro-4-oxo-1,2,3-benzotriazine (HOOBt).

It is preferred to apply DIC or TCTU as coupling reagents and HOBt or 6-chloro-HOBt as coupling additives.

According to the present invention, the amount of each amino acid or peptide used in steps (b) and (c) is between 1 and 3 equivalents. Preferably N-terminally protected Arg or Har is used in amounts between 1.5 and 2.5 equivalents.

The solid-phase support may be any commonly employed solid-phase resin, preferably an activated halogen, an activated derivative of hydroxy or carboxy functionalized resin or grafted linker-resin composite. The polymer matrix of the resin may be e.g. polystyrene, polyethylene-glycol (PEG), cross-linked PEG, polyamide, polyvinylalcohol (PVA) or polyoxyalkylene. It may be pure or mixed resin, including block-copolymers or grafted resins such as PVA grafted on PEG resin, PEG-grafted polystyrene-divinylbenzene (PS-DVB) resins, polyoxyethylene resins grafted onto an inner polystyrene matrix, wherein the functionalized groups for coupling being exposed on the polyoxyethylene branches.

Common examples are 2-chlorotrityl chloride polystyrene (2-CTC) resin, bromo-(4-methyl-phenyl)-methyl polystyrene resin, bromo-(4-methoxyphenyl)-methyl polystyrene resin, Merrifield resin or Wang resin.

In a preferred embodiment of the present invention A is formed from an activated grafted linker-resin composite selected from the group consisting of 2-chlorotrityl chloride polystyrene resin, bromo-(4-methylphenyl)-methyl polystyrene resin, bromo-(4-methoxyphenyl)-methyl polystyrene resin and activated hydroxy-(4-methylphenyl)-methyl polystyrene resin.

In a preferred embodiment, the peptides obtained by the method of the present invention are Trp-Arg-Arg-Lys-NH₂, Trp-Trp-Pro-Trp-Arg-Arg-Lys-NH₂ or Ile-Leu-Arg-Trp-Pro-Trp-Trp-Pro-Trp-Arg-Arg-Lys-NH₂.

Another object of the present invention is to provide a compound of formula

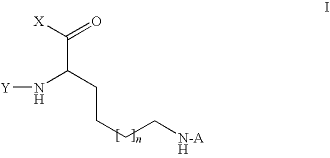

wherein A is a solid-phase support or a linker grafted to a solid-phase support; n is an integer between zero and ten; X is C₁₋₆ alkoxy, aryl-substituted C₁₋₆ alkoxy, aryloxy, allyloxy, an optionally protected amino acid residue, an optionally protected peptide residue or NR¹R², wherein R¹ and R² are independently hydrogen or C₁₋₁₀ alkyl; and Y is a protecting group being orthogonal to the bond between A and the amino function, or an optionally further protected α-amino protected or unprotected amino acid or peptide residue.

The compound of formula I is useful as intermediate in the method of the invention.

In a preferred embodiment, Y of the compound of formula I is an orthogonal protecting group selected from the group consisting of Fmoc, Boc, Cbz, Npys and Alloc; with the proviso that Y is not Alloc if X is allyloxy.

Preferred is a compound of formula I, wherein n is an integer between zero and ten.

In an also preferred embodiment, X of the compound of formula I is NR¹R² with R¹ and R² are independently hydrogen or C₁₋₁₀ alkyl; and Y is Fmoc, Boc, Cbz, Npys or Alloc.

A further preferred embodiment is the compound of formula I, wherein Y is an α-amino protected or unprotected amino acid residue or an optionally further protected peptide residue selected from the group consisting of Y′-Ile-Leu-Arg-Trp-Pro-Trp-Trp-Pro-Trp-Arg-Arg, Y % Trp-Trp-Pro-Trp-Arg-Arg, Y′-Trp-Arg-Arg, Y′-Arg-Arg and Y′-Arg, wherein Y′ is hydrogen or a suitable protecting group, and wherein the amino acid residues are optionally protected at their side chains with suitable protecting groups.

Another object of the present invention is a compound of formula

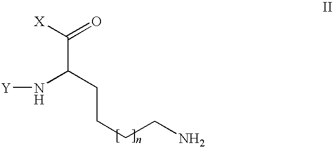

wherein n is an integer between zero and ten; X is C₁₋₆ alkoxy, aryl-substituted C₁₋₆ alkoxy, aryloxy, allyloxy or NR¹R², wherein R¹ and R² are independently hydrogen or C₁₋₁₀ alkyl; Y is Fmoc, Boc, Cbz, Npys, Alloc, an α-amino protected or unprotected amino acid residue or an optionally further protected peptide residue; with the proviso that Y is not Alloc if X is allyloxy.

### EXPERIMENTS

The following examples further illustrate this invention but are not intended to limit it in any way.

### Example 1

### Solid-Phase Synthesis of Ile-Leu-Arg-Trp-Pro-Trp-Trp-Pro-Trp-Arg-Arg-Lys-NH₂

All amino acids used in example 1 were of L configuration. 33 g of H-Lys(Boc)-NH₂ (from Genzyme) was converted to Fmoc-Lys(Boc)-NH₂ by reaction with Fmoc-chloride and 10% Na₂CO₃ in dioxane/water (1:1). Then, the side chain protecting Boc group was removed at ambient temperature with 50% trifluoroacetic acid (TFA) in dichloromethane. After addition of methyl tert-butyl ether, the Fmoc-Lys-NH₂ precipitated as its TFA salt. The salt was recovered, dissolved in aqueous basic media and subsequently extracted, affording salt-free Fmoc-Lys-NH₂ dissolved in the organic phase.

30 g of 2-chlorotrityl chloride polystyrene resin (from CBL-Patras) was added to the organic phase of the preceding extraction and stirred in the presence of an organic base, preferably diisopropylethylamine. After reaction of the ε-amino group with the resin, its loading was about 0.50 mmol/g, yielding the compound of formula

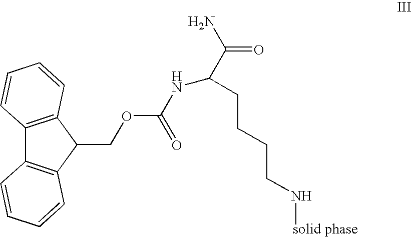

Then, the compound of formula III was deprotected by reaction with 20% piperidine. A mixture of 2 equivalents of Fmoc-Arg(Pbf)-OH, 1 equivalent of HOBt and 1 equivalent of diisopropyl-carbodiimide was prepared in N-methylpyrrolidone and added to the deprotected amino acid resin. After a coupling period between 60 and 90 minutes at ambient temperature, a coupling efficiency of ≧99% was achieved without any further repetition of the procedure.

The dipeptide resin was washed with N-methylpyrrolidone and the further amino acids were sequentially assembled at ambient temperature using 2 equivalents each of the respective Fmoc-amino acid, with the exception of the last amino acid which was Boc-Ile-OH, in the presence of 1 equivalent of 6-chloro-HOBt, TCTU and diisopropylethylamine in dichloromethane for a coupling time of 30-60 minutes. The washes were performed with N-methylpyrrolidone. Each coupling step was only done once, i.e. no repetition of individual coupling steps took place. After coupling of the last amino acid the peptide resin Boc-Ile-Leu-Arg(Pbf)-Trp(Boc)-Pro-Trp(Boc)-Trp(Boc)-Pro-Trp(Boc)-Arg(Pbf)-Arg(Pbf)-Lys(solid-phase)-NH₂ was obtained which was deprotected and removed from the resin by treatment with a mixture of TFA 60%, thioanisole 5%, phenol 5%, triisopropylsilane (TIS) 1%, dithiothreitol (DTT) 2.5%, water 5% and dichloromethane 21.5%, yielding 28.2 g of crude Ile-Leu-Arg-Trp-Pro-Trp-Trp-Pro-Trp-Arg-Arg-Lys-NH₂ as white solid (structure confirmed by MS).

### Example 2

### Solid-Phase Synthesis of Ile-Leu-Arg-Trp-Pro-Trp-Trp-Pro-Trp-Arg-Arg-Lys-NH₂

All amino acids used in example 3 were of L configuration. 54 g of H-Lys(Boc)-NH₂×HCl (from Genzyme) was converted to Alloc-Lys(Boc)-NH₂ by reaction with Alloc-OSu (allyloxycarbonyloxysuccinimide) and triethylamine in dioxane. Then, the side chain protecting Boc group was removed at 0° C. with hydrogen chloride gas in dichloromethane. After reaction, Alloc-Lys-NH₂ precipitated as HCl salt. It was filtered and washed with dichloromethane. 50 g of bromo-(4-methylphenyl)-methyl polystyrene resin (from CBL-Patras) and 31.7 g of Alloc-Lys-NH₂×HCl were coupled at elevated temperature in the presence of diisoproylethylamine in N-methylpyrrolidone. After reaction of the s-amino group with the resin, its loading was about 0.55 mmol/g, yielding the compound of formula

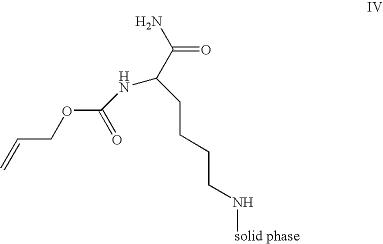

Then, the compound of formula IV was deprotected by treatment with Pd[PPh₃]₄ in N,N-dimethylformamide and in the presence of sodium p-toluenesulfinate. The further steps were performed analogous to example 1, except for the coupling mixture consisting of 1 equivalent of HOBt and 1 equivalent of diisopropylcarbodiimide in N-methylpyrrolidone for each coupling step. Yield: 41.3 g of Ile-Leu-Arg-Trp-Pro-Trp-Trp-Pro-Trp-Arg-Arg-Lys-NH₂ (structure confirmed by MS).

Similar to example 1, the Fmoc-Arg(Pbf) was coupled to the deprotected amino acid resin of formula IV with an efficiency of >99% in only one coupling step.

### Comparison Example C1

### Solid-Phase Synthesis of Ile-Leu-Arg-Trp-Pro-Trp-Trp-Pro-Trp-Arg-Arg-Lys-NH₂

The procedure of example 1 was repeated except for anchoring the first amino acid residue traditionally via its C-terminus to the resin, thus affording Fmoc-Lys(Boc)-solid-phase. The following coupling with Fmoc-Arg(Pbf) required a substantially longer coupling time (8 hours) and repetition of the coupling step with 4 equivalents of Fmoc-Arg(Pbf) per cycle for at least two times.

1. A method for peptide synthesis starting from a compound of formula 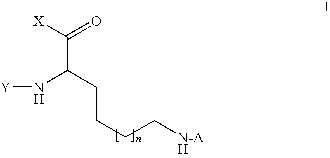
wherein A is a solid-phase support or a linker grafted to a solid-phase support; n is an integer between zero and ten; X is C₁₋₆ alkoxy, aryl-substituted C₁₋₆ alkoxy, aryloxy, allyloxy, an optionally protected amino acid residue, an optionally protected peptide residue or NR¹R², wherein R¹ and R² are independently hydrogen or C₁₋₁₀ alkyl; and Y is a protecting group being orthogonal to the bond between A and the amino function; and comprising the steps of
(a) deprotecting the N-terminal α-amino function,
(b) coupling an at least N-terminally protected amino acid or peptide having a free or activated carboxylic acid function with the deprotected α-amino function of step (a), thus elongating the compound of formula I,
(c) optionally repeating at least once steps (a) and (b), wherein the at least N-terminally protected amino acid or peptide is identical or different to that of the preceding step (b),
(d) cleaving the resulting peptide from A,
(e) optionally removing all protecting groups which remained after step (d),
(f) isolating and optionally purifying the peptide thus obtained.

2. The method of claim 1, wherein Y is an orthogonal protecting group selected from the group consisting of Fmoc, Boc, Cbz, Npys and Alloc; with the proviso that Y is not Alloc if X is allyloxy.

3. The method of claim 1, wherein n is an integer between zero and four; and R¹ and R² are independently hydrogen, methyl or ethyl.

4. The method of claim 1, wherein n is one; X is NR¹R², wherein both R¹ and R² are hydrogen; and Y is Fmoc or Alloc.

5. The method of claim 1, wherein the N-terminally protected amino acid of step (b) is N-terminally protected Arg or Har; or wherein the N-terminally protected peptide of step (b) contains Arg or Har as C-terminal residue.

6. The method of claim 1, wherein Y is Fmoc or Alloc, and wherein the N-terminally protected amino acids or peptides of steps (b) and (c) are Fmoc-protected.

7. The method of claim 6, wherein the at least N-terminally protected amino acid or peptide of the lastly repeated step (c) is protected by an protecting group which is orthogonal to Fmoc.

8. The method of claim 7, wherein the orthogonal protecting group is Boc.

9. The method of claim 1, wherein A is an activated grafted linker-resin composite selected from the group consisting of 2-chlorotrityl chloride polystyrene resin, bromo-(4-methylphenyl)-methyl polystyrene resin and bromo-(4-methoxy-phenyl)-methyl polystyrene resin.

10. The method of claim 1, wherein the peptide obtained in step (f) is Ile-Leu-Arg-Trp-Pro-Trp-Trp-Pro-Trp-Arg-Arg-Lys-NH₂.

11. The method of claim 1, wherein the peptide obtained in step (f) is Trp-Trp-Pro-Trp-Arg-Arg-Lys-NH₂.

12. The method of claim 1, wherein the peptide obtained in step (f) is Trp-Arg-Arg-Lys-NH₂.

13. A compound of formula 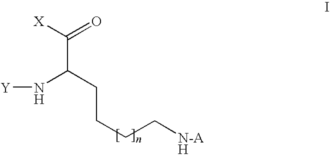
wherein A is a solid-phase support or a linker grafted to a solid-phase support; n is an integer between zero and ten; X is C₁₋₆ alkoxy, aryl-substituted C₁₋₆ alkoxy, aryloxy, allyloxy, an optionally protected amino acid residue, an optionally protected peptide residue or NR¹R², wherein R¹ and R² are independently hydrogen or C₁₋₁₀ alkyl; and Y is a protecting group being orthogonal to the bond between A and the amino function, or an optionally further protected α-amino protected or unprotected amino acid or peptide residue.

14. The compound of claim 13, wherein Y is an orthogonal protecting group selected from the group consisting of Fmoc, Boc, Cbz, Npys and Alloc; with the proviso that Y is not Alloc if X is allyloxy.

15. The compound of claim 13, wherein n is an integer between zero and ten.

16. The compound of claim 13, wherein X is NR¹R² with R¹ and R² are independently hydrogen or C₁₋₁₀ alkyl; and Y is Fmoc, Boc, Cbz, Npys or Alloc.

17. The compound of any of claim 13, wherein Y is an α-amino protected or unprotected amino acid residue or an optionally further protected peptide residue selected from the group consisting of Y′-Ile-Leu-Arg-Trp-Pro-Trp-Trp-Pro-Trp-Arg-Arg, Y′-Trp-Trp-Pro-Trp-Arg-Arg, Y′-Trp-Arg-Arg, Y′-Arg-Arg and Y′-Arg, wherein Y′ is hydrogen or a suitable protecting group and wherein the amino acid residues are optionally protected at their side chains with suitable protecting groups.

18. A compound of formula 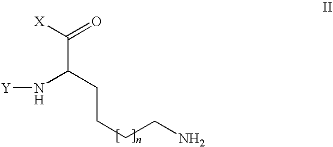
wherein n is an integer between zero and ten; X is C₁₋₆ alkoxy, aryl-substituted C₁₋₆ alkoxy, aryloxy, allyloxy or NR¹R², wherein R¹ and R² are independently hydrogen or C₁₋₁₀ alkyl; Y is Fmoc, Boc, Cbz, Npys, Alloc, an α-amino protected or unprotected amino acid residue or an optionally further protected peptide residue; with the proviso that Y is not Alloc if X is allyloxy.
